(19) United States
(12) Patent Application Publication
(10) Pub. No.: US 2025/0025216 A1
(43) Pub. Date: Jan. 23, 2025

(54) FUSION CAGES AND METHODS FOR SACRO-ILIAC JOINT STABILIZATION

(71) Applicant: SI-Bone Inc., Santa Clara, CA (US)

(72) Inventors: Ali H. MESIWALA, Claremont, CA (US); Francois FOLLINI, Austin, TX (US); Bret W. SCHNEIDER, San Jose, CA (US); Scott A. YERBY, Montara, CA (US)

(21) Appl. No.: 18/716,090

(22) PCT Filed: Dec. 2, 2022
(86) PCT No.: PCT/US2022/080830

§ 371 Date: Jun. 3, 2024

Related U.S. Application Data
(60) Provisional application No. 63/264,921, filed on Dec. 3, 2021.

(51) Int. Cl. A61B 17/70; A61F 2/30; A61F 2/44

(52) U.S. Cl. CPC A61B 17/7055; A61F 2/4455; A61F 2002/30995

(57) ABSTRACT

Systems, devices and methods for vertebral fusion and SI joint stabilization.

### CROSS REFERENCE TO RELATED APPLICATIONS AND INCORPORATION BY REFERENCE

This application claims priority to U.S. Provisional Application No. 63/264,921, filed Dec. 3, 2021, the entire disclosure of which is incorporated by reference herein for all purposes.

All publications and patent applications mentioned in this specification are herein incorporated by reference to the same extent as if each individual publication or patent application was specifically and individually indicated to be incorporated by reference.

### BACKGROUND

There is a need for systems and methods that can provide vertebral fusion (e.g., fusing L5-S1 vertebrae) as well as sacro-iliac (SI) joint stabilization or fusion.

### SUMMARY

One aspect of the disclosure is a method of fusing vertebrae and stabilizing sacro-iliac joints of a subject. The method may include implanting at least a portion of a fusion cage between an S1 vertebra and an L5 vertebra, optionally fully between an S1 vertebra and an L5 vertebra. The method may also include implanting a first SI joint stabilizing member such that it interfaces with the fusion cage, and also extends across a first SI joint and into a first ilium. The method may also include implanting a second SI joint stabilizing member such that it interfaces with the fusion cage, and also extends across a second SI joint and into a second ilium.

One aspect of the disclosure is a system for fusing vertebrae and stabilizing sacro-iliac joints of a subject. The system may include a fusion cage sized and configured to be at least partially implanted between an S1 vertebra and an L5 vertebra.

In this aspect, the system may also include one or more of a first SI joint stabilizing member or a second SI joint stabilizing member.

In this aspect, a fusion cage may include a first SI joint stabilizing member interface in an anterior portion of the fusion cage and in a first lateral portion of the fusion cage, and a second SI joint stabilizing member interface in the anterior portion of the fusion cage and in a second lateral portion of the fusion cage. SI joint stabilizing member interfaces, in this aspect, may have at least one surface that is sized, configured and positioned to stably interface with a cage end region of an SI joint stabilizing member when the SI joint stabilizing member extends posterior, caudal and lateral relative to the fusion cage such that an ilium end of the SI joint stabilizing member is implanted in an ilium of the patient.

This aspect may include any other suitably combinable aspect described herein, including any of the cages, stabilizing members, and/or methods of use.

One aspect of this disclosure is fusion cages that is adapted for SI joint stabilization. The fusion cages may include a body sized and configured to be at least partially implanted between an S1 vertebra and an L5 vertebra. The fusion cages may also include one or more SI joint stabilizing member interfaces in an anterior portion of the cage body and in a lateral portion of the fusion cage. The fusion cage may also include one or more L5 stabilizing member interfaces, optionally disposed along a midline of the fusion cage measured in a width dimension. Fusion cages in this aspect may include any other suitably combinable aspect described herein, including any of the cages, stabilizing members, and/or methods of use.

### BRIEF DESCRIPTION OF THE FIGURES

FIG. 1 is a perspective view illustrating a lower portion of the spine, a portion of a sacrum, iliac bones, and SI joints.

FIG. 2 is an example of a fusion system.

FIG. 3A shows an exemplary fusion system is an exemplary implanted position.

FIG. 3B shows an exemplary fusion system is an exemplary implanted position.

FIG. 3C shows an exemplary fusion system is an exemplary implanted position.

FIG. 3D shows an exemplary fusion system is an exemplary implanted position.

FIGS. 4A, 4B, 4C and 4D illustrate exemplary SI joint stabilizing members and optional pins.

FIG. 5 is atop view illustrating an exemplary fusion system implanted, including SI joint stabilizing members implanted across both SI joints.

FIGS. 6A, 6B and 6C illustrate fusion and SI joint stabilization when a fusion cage is implanted at least partially in the L5 vertebra.

FIG. 7A illustrates an anterior view of an exemplary fusion cage with a plurality of covering members in uncovered/unlocked positions.

FIG. 7B illustrates a side view of an exemplary fusion cage with a plurality of covering members in uncovered/unlocked positions.

FIG. 7C illustrates a top view of an exemplary fusion cage with a plurality of covering members in uncovered/unlocked positions.

FIG. 7D illustrates a perspective posterior view of an exemplary fusion cage with a plurality of covering members in uncovered/unlocked positions.

FIG. 7E illustrates a perspective anterior view of an exemplary fusion cage with a plurality of covering members in uncovered/unlocked positions.

FIG. 8A illustrates an exemplary fusion cage with a plurality of covering members in uncovered/unlocked positions.

FIG. 8B illustrates an exemplary fusion cage with a plurality of covering members in covered/locked positions.

FIG. 8C illustrates an exemplary fusion cage with a plurality of covering members in uncovered/unlocked positions.

FIG. 8D illustrates an exemplary fusion cage with a plurality of covering members in covered/locked positions.

FIG. 9 illustrates exemplary SI joint stabilizing members implanted across SI joints.

FIG. 10 is an example of an SI joint stabilizing member.

### DETAILED DESCRIPTION

This disclosure describes methods, systems and devices that facilitate vertebral fusion, as well as SI joint stabilization or fusion. Some particular and exemplary embodiments include fusing the L5 and S1 vertebrae, as well as stabilizing both SI joints. In some embodiments, the systems include a fusion cage and a plurality of stabilizing members adapted to interface with the fusion cage. In general, the one or more stabilizing members are sized and configured to stabilize the fusion cage relative to the vertebrae and stabilize the SI joint(s).

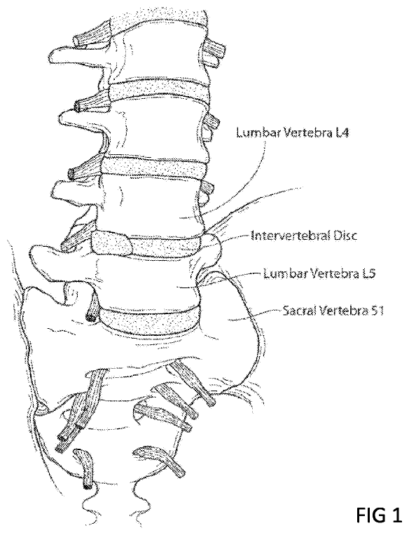

FIG. 1 is a perspective view that illustrates a lower portion of the spine, a portion of the sacrum and iliac bones, and the SI joints. In some embodiments herein, a fusion cage is implanted into the space between the L5 vertebra and the S1 vertebra, which is shown in FIG. 1, exemplary general methods of which are known.

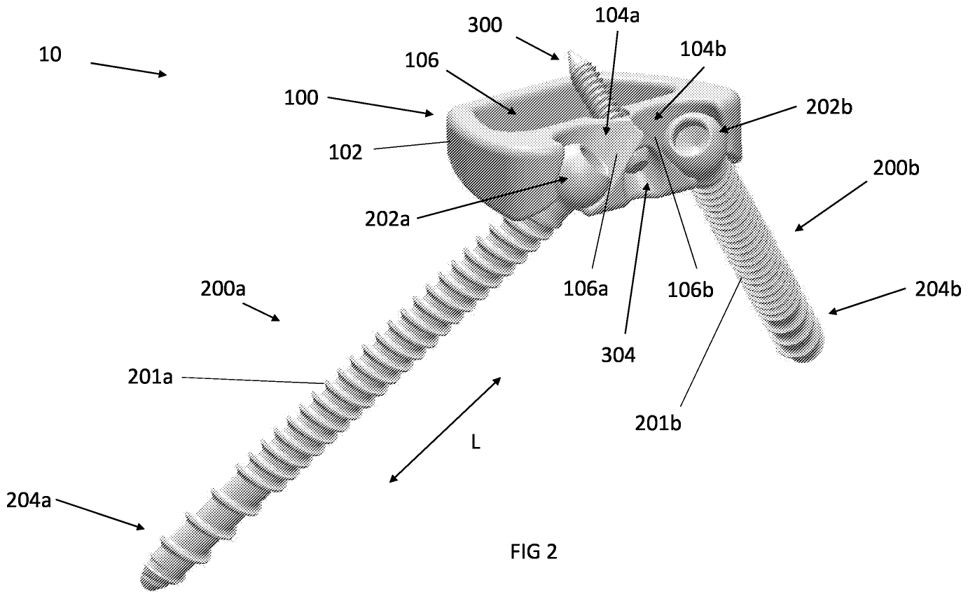

FIG. 2 illustrates an exemplary vertebral fusion and SI joint stabilization system 10, which includes fusion cage 100 and SI joint stabilizing members 200a and 200b. In this example, the SI joint stabilizing members are threaded implants and include one or more threads, as shown. FIG. 2 is a perspective view of the system, generally anterior, also illustrating a superior or top portion or side of the cage body 102 (inferior or bottom portion not visible). Fusion cage 100 includes cage body 102, which includes first and second SI joint stabilizing member interfaces 104a and 104b in an anterior portion of the cage body, as shown. The first SI joint stabilizing member interface 104a includes at least one surface 106a, as shown, configured to engage with a cage end region 202a of first SI joint stabilizing member 200a when the fusion cage is disposed between an L5 vertebra and an S1 vertebrae of a sacrum and when first SI joint stabilizing member 200a is implanted in a caudal, lateral and posterior direction across a first SI joint of the patient with an ilium end region 204a of first SI joint stabilizing member 200a implanted in a first ilium of the patient. The second SI joint stabilizing member interface 104b includes at least one surface 106b, as shown, configured to engage with cage end region 202b of second SI joint stabilizing member 200b when the fusion body is disposed between the L5 vertebra and the S1 vertebrae of a sacrum and when second SI joint stabilizing member 200b is implanted in a caudal, lateral and posterior direction across a second SI joint of the patient with ilium end region 204b of the second SI joint stabilizing member implanted in a second ilium of the patient.

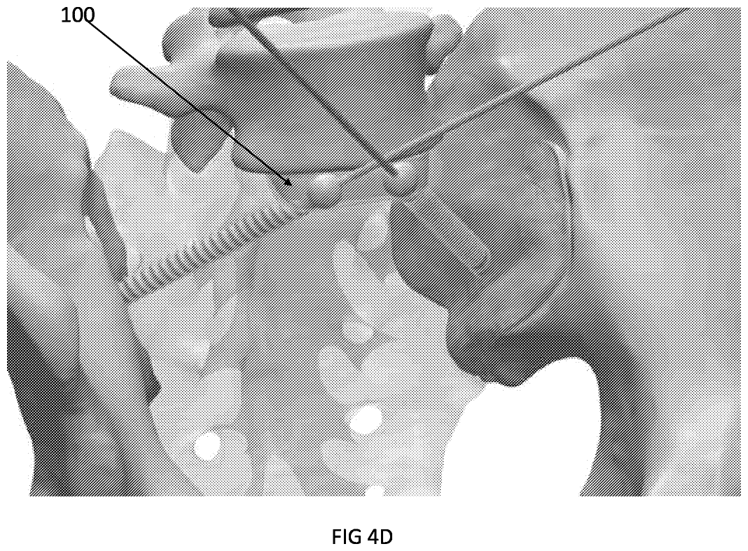

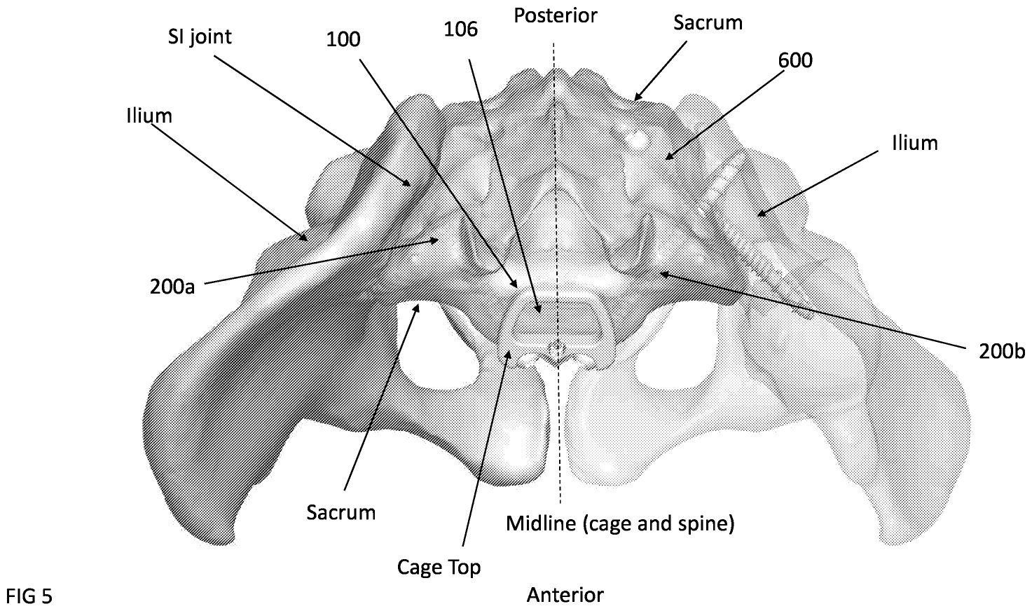

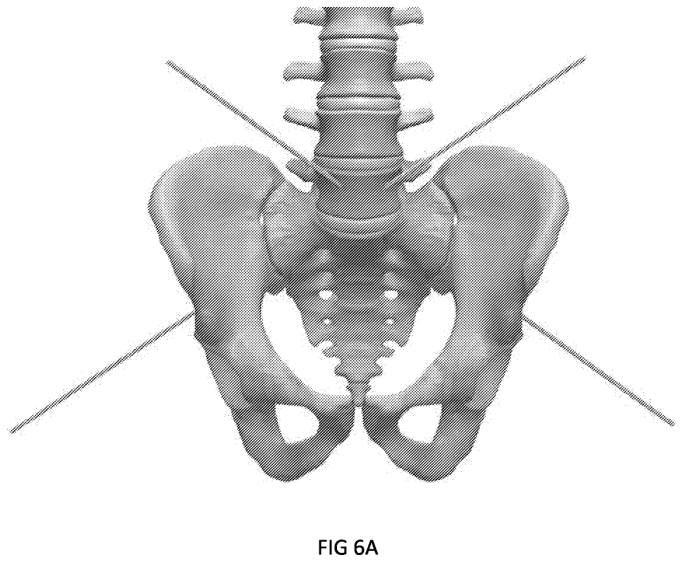

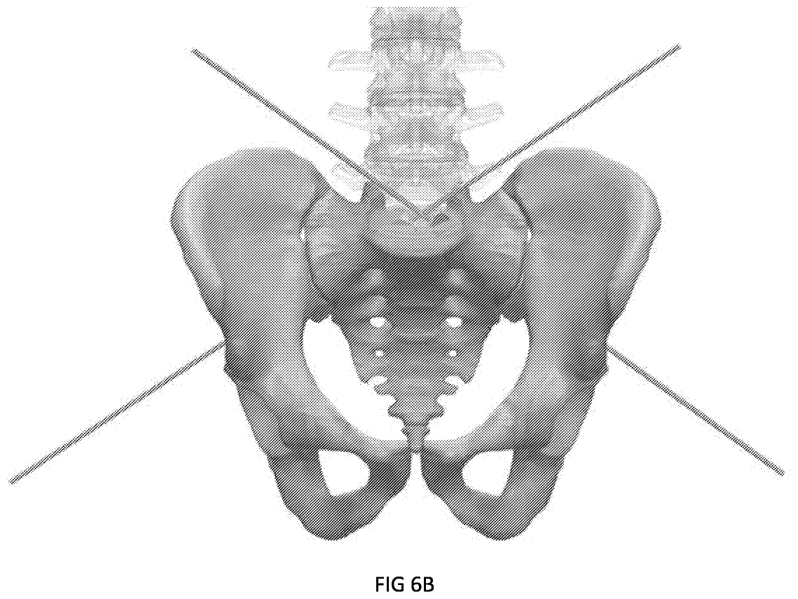

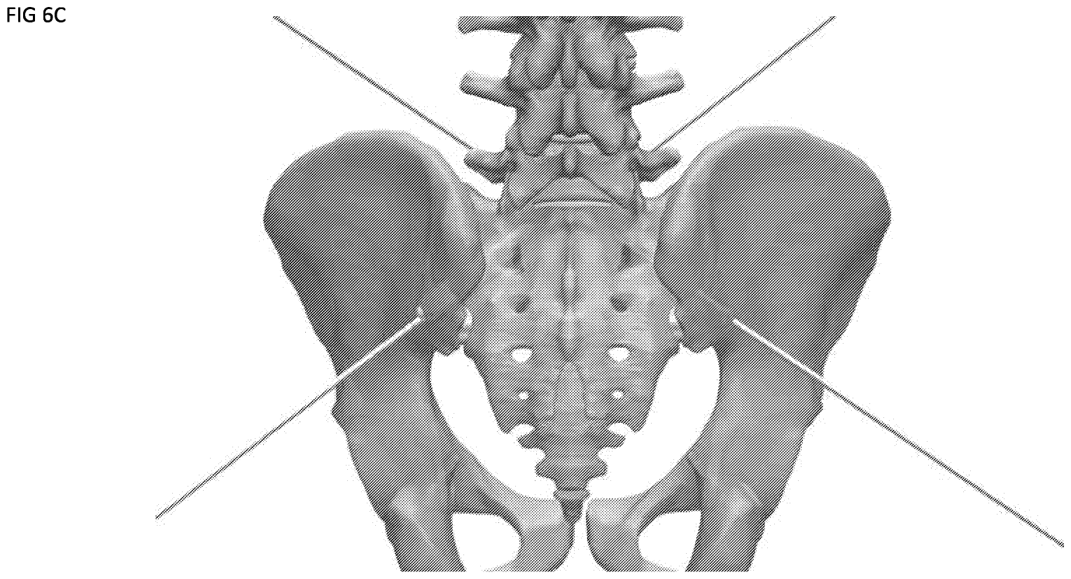

Cage body 102 (also shown in FIG. 7C) is also an example of a fusion cage body that comprises a first SI joint stabilizing member interface (e.g., 104a) in a first lateral portion 731a of the fusion cage and a second SI joint stabilizing member interface (e.g., 104b) in a second lateral portion 731b of the fusion cage. In this context, the lateral portions 731a and 731b refer generally to the entire lateral portions of the cage on either side of an imaginary midline (the dotted line in FIG. 7C), the midline measured along the Width of the implant (the width dimension is illustrated in FIG. 7C). In this example, cage body 102 includes SI joint stabilizing member interfaces that are also disposed in an anterior portion of the cage, as shown herein. In this example, cage body 102 includes SI joint stabilizing member interfaces that also exit from or pass through the inferior or caudal surface of the cage, as shown herein.

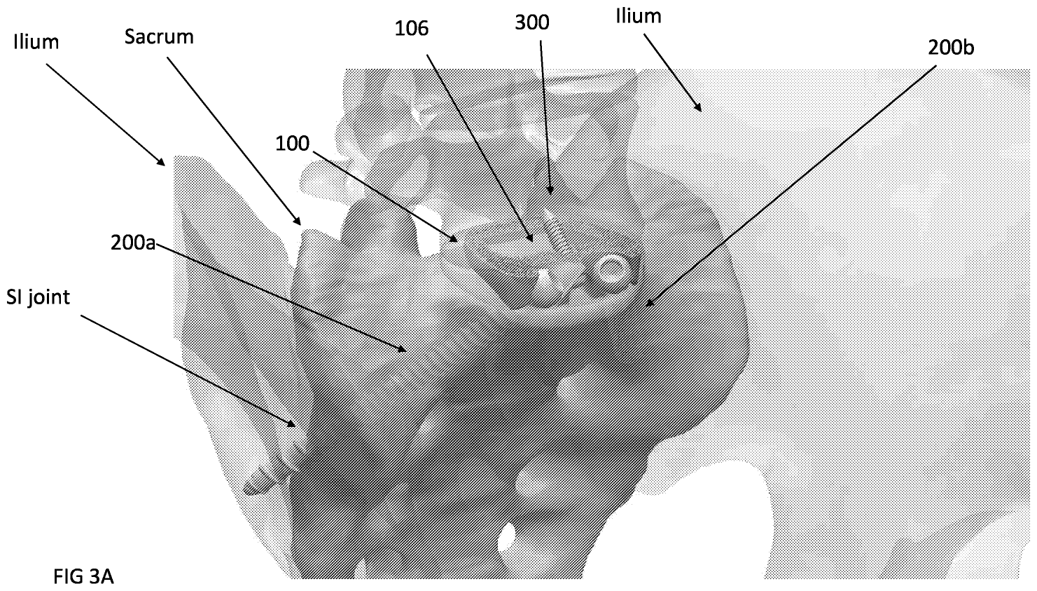

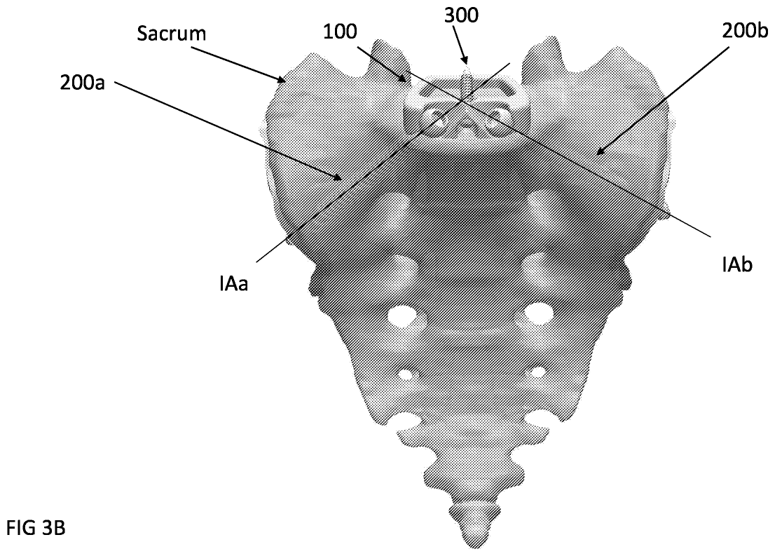

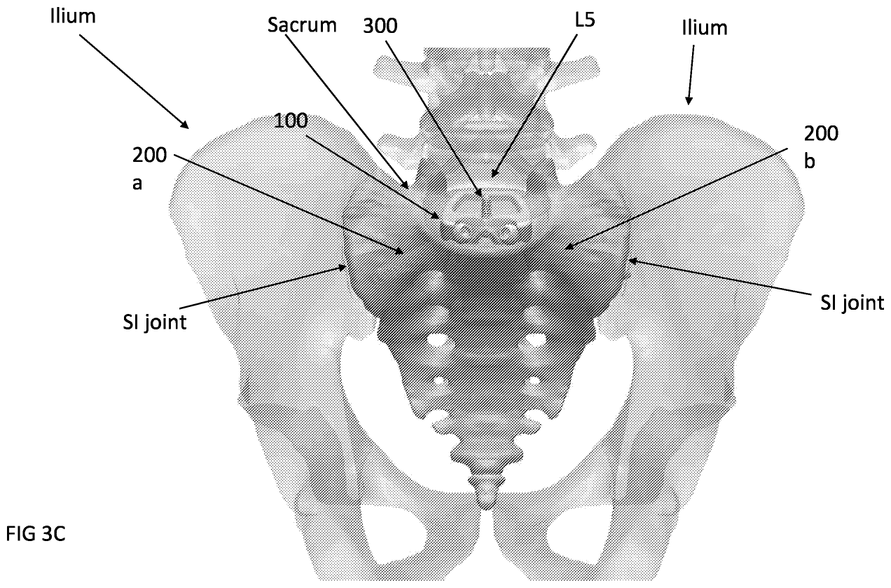

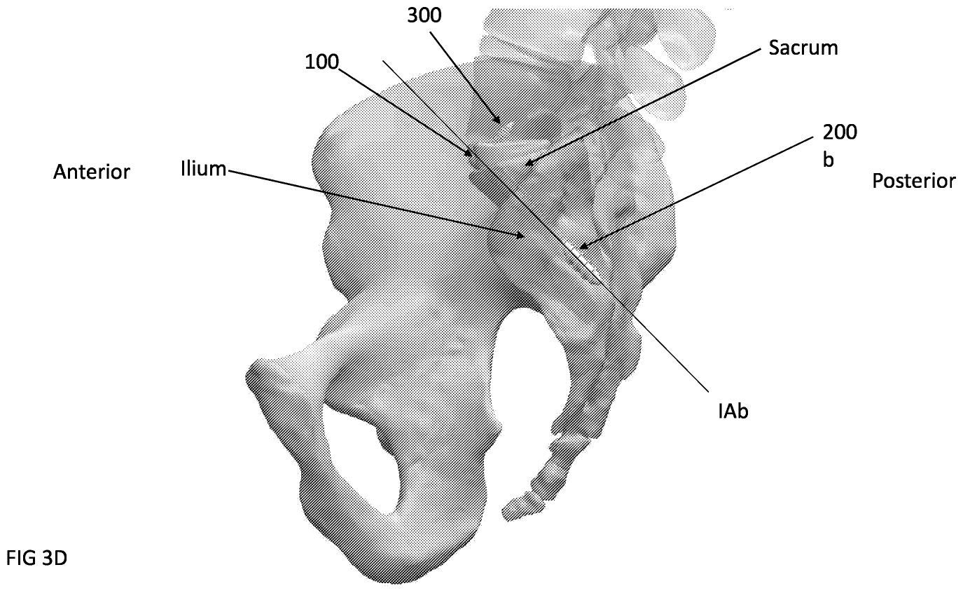

The body (e.g., body 102) of the fusion cages herein (e.g., cages 100, 700) may have a variety of configurations, and known lumbar fusion cages are incorporated by reference herein as examples in this regard. For example, the body of the cages herein may have a general wafer configuration (width greater than height), of which body 102 in FIG. 2 is an example. The fusion cages herein may also have a wedge configuration, having a height in an anterior side that is greater than a height in the posterior side, as can be more clearly seen in the lateral view of implantation of FIG. 3D. The cages herein will generally include an opening therethrough in a central region, such as opening 106 shown in FIG. 2, which extends through top and bottom surfaces of cage body 102, to allow growth therethrough. Cage 100 (and cage 700) is an example of a cage with a cage body having top and bottom surfaces, lateral sides, an anterior side, and a posterior side. The term side in this context does not require flat surfaces, but rather refers generally to the relative position of the side(s), and includes sides with surfaces having non-flat configurations, such as surfaces at least a portion of which is curved. In some embodiments the top and bottom of the cage body may have flat or substantially flat surfaces, which may not be in parallel planes if the cage is wedged shaped. Additionally, the terms anterior and posterior in this context refer to the anterior and posterior directions relative to a patient when implanted.

The fusion cages herein may be made of a variety of materials, such as one or more metals, polymers, ceramics, including any combination thereof. In some embodiments, the cages herein be made from titanium or polyetheretherketone (“PEEK”). The cages may optionally be manufactured using additive manufacturing techniques, such as incorporating 3D printing processes. The cages herein may optionally include a porous coating on one or more surfaces thereof, such as the top surface of the implant, as is illustrated in implant 100 in exemplary FIG. 3A.

Any of the fusion cages herein may optionally be used or have application in the treatment of one or more of degenerative disc disease, spondylolisthesis, spinal tumors or masses, spinal stenosis, scoliosis, and/or herniated discs, in addition to stabilizing SI joints, which is described throughout this disclosure.

The fusion cages herein include first and second SI joint stabilizing member interfaces (e.g., 104a and 104b in FIG. 2). Each stabilizing member interface includes at least one surface that is configured to allow the stabilizing member to be implanted caudal, lateral and posterior relative to the cage, and wherein the interface is configured to engage (optionally stably engage) a cage end region (e.g., 202a, 202b) of the SI joint caudal stabilizing members, such as a rounded proximal end of the SI joint caudal stabilizing members. Exemplary axes of the SI joint stabilizing members when implanted across the SI joints and into the ilia are illustrated in FIGS. 3B and 3D, and as shown the axes generally extend caudal, lateral, and posterior relative to the fusion cage body when the fusion cage is in place and implanted between the L5 and S1 vertebrae. The SI joint stabilizing member interfaces of the cage bodies herein are specifically configured to facilitate and allow this type of stabilization member orientation and trajectory relative to the cage body, and optionally to facilitate implantation along these trajectory/axes if the cage is implanted at least partially between the L5 and S1 vertebrae. This is in contrast to fusion cages that are not adapted and configured in this manner to allow a stabilizing member to interface with the fusion cage (when placed between the L5-S1 vertebrae) and extend across an SI joint. Additionally, the SI joint stabilizing member interfaces are configured to interface or engage with the cage end regions of the stabilizing members when the stabilizing members are in their final implanted positions in which they extend across the SI joints and into the ilia, as shown generally in FIGS. 3A-3D. In particular, the interfaces (104a, 104b) each include at least one surface that is configured to interface with a cage end region (e.g. 202a, 202b) of one of the SI joint stabilizing members (e.g., 200a, 200b) when the fusion cage is disposed between an L5 vertebra and a sacrum and when the SI joint stabilizing members are fully implanted and extend caudal, lateral, and posterior (relative to the fusion cage) across the SI joints with an ilium end region of each of the SI joint stabilizing members extending into and implanted in an ilium of the patient.

The stabilizing member interfaces (e.g., 104a, 104b) of the fusion cages herein may be considered to have a channel configuration (which may be seen most easily in FIGS. 3B and 7A-8D) that is generally oriented caudal, lateral and posterior relative to top and bottom surfaces of the cage body, which facilitates the desired placement of the SI joint stabilizing members and their resulting axes once implanted. The channels herein may be considered to have axes that are caudal, lateral and posterior relative to top and bottom surfaces of the cage body. In this context, the channels are at least partially open or exposed on a portion of the anterior sides, as shown in FIGS. 3A, 3B, 3C and 3D, but may not be open on inferior (bottom) regions of the channel, which is shown in FIGS. 7A-7E. Alternatively stated, the inferior or bottom end of the channels may define an aperture or hole, such as holes 787 labeled in FIGS. 7C and 7E. In these examples, as shown, the SI joint stabilizing member interfaces generally taper in their inner dimensions towards the inferior side of the cage, such that the end cage region of the SI joint stabilizing member interfaces with at least one surface of the interface and cannot pass through the interface. For example, a circular head on the stabilizing members may interface with the generally annular surfaces of the interface that define holes 787.

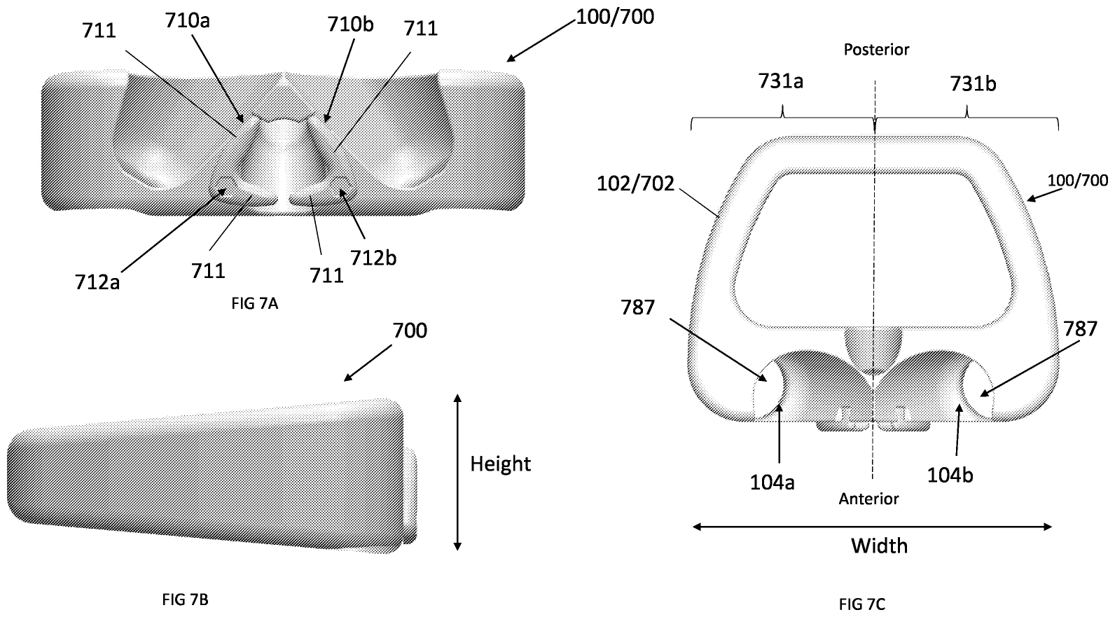

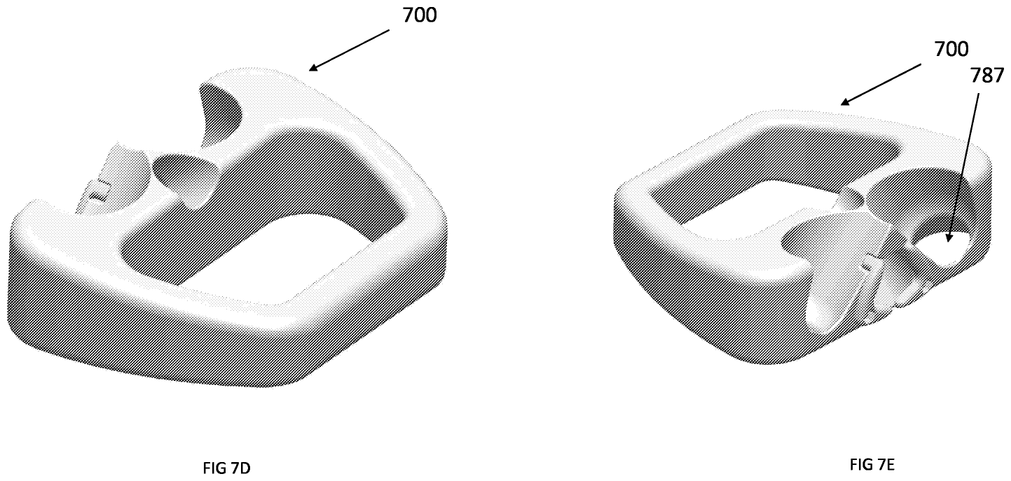

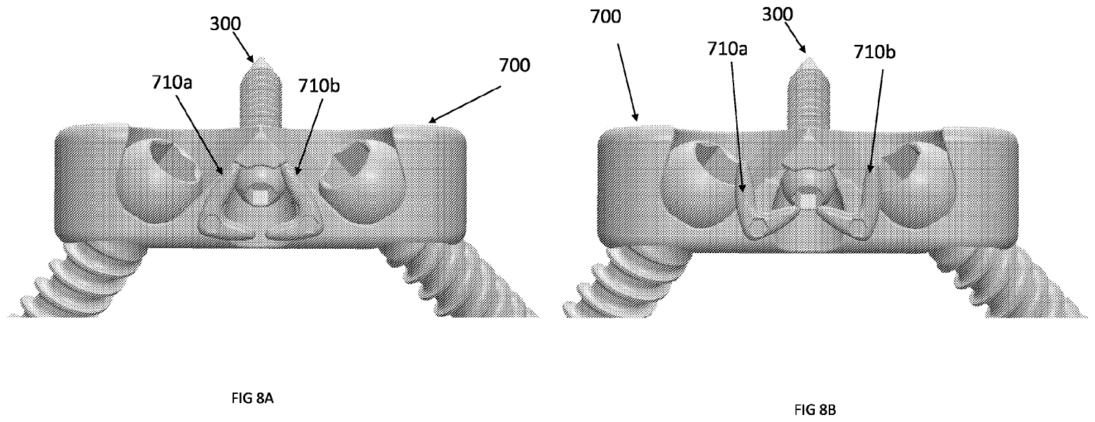

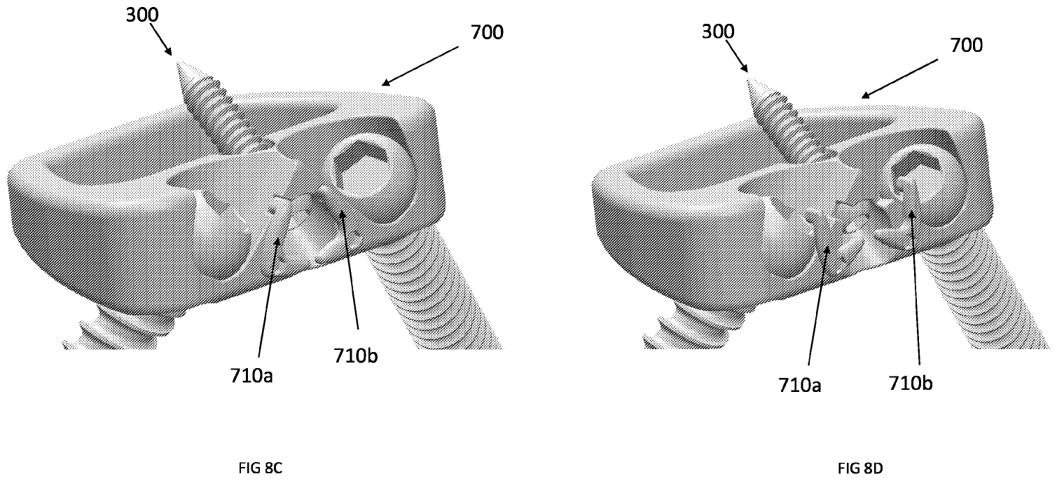

The interfaces may define larger open areas (define more open space) in the top or superior region of the interface than in the bottom or inferior regions (shown in FIGS. 7A-8D), which may allow for the stabilizing members to be advanced through the interfaces at slightly different angles relative to the cage body depending on the desired particular implant location relative to the fusion cage. This may thus allow for some patient-to-patient anatomical variability. Additionally, the implantation axes of the two stabilizing members may not necessarily be symmetrical about a midline of the fusion cage, and the interfaces may thus optionally be configured to accommodate slightly different stabilizing member trajectories and implant positions relative to the fusion cage.

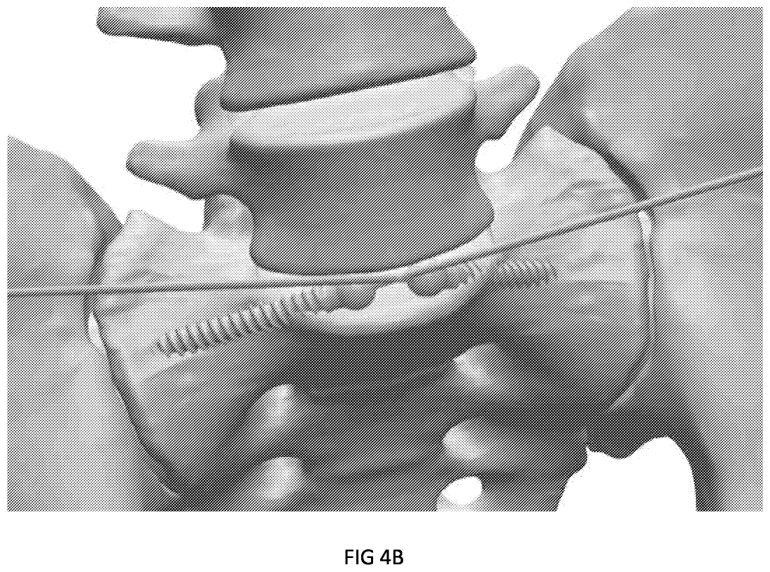

The channel configuration and axes defined by the stabilizing member interfaces may be symmetrical about a midline of the fusion cage (exemplary midline illustrated in FIG. 5), and they may be oriented generally laterally, caudal and posterior relative to the midline (as shown in FIGS. 3A-3D). The midline of the fusion cage body in this context is measured across a width of the body, as shown in FIG. 5.

In any of the embodiments, the systems may further include one or more covers or covering features coupled to or adapted to be coupled to the anterior side of the cage and/or the proximal regions of the stabilizing member to at least partially cover and/or provide more stability to the cage end regions of the stabilizing members and reduce stabilizing member back-out relative to the fusion body. For example only, the system may optionally include a locking plate configured to interface with the anterior side of the cage, or one or more washers adapted to be disposed over a proximal region of the stabilizing members.

FIGS. 7A, 7B, 7C, 7D, 7E, 8A, 8B, 8C and 8D illustrate exemplary fusion cage 700 and exemplary stabilizing members (which may have any of the same features as the stabilizing members in FIGS. 1-5 and the descriptions thereof), wherein the cage includes first and second covering members, each adapted to be movable relative to an anterior side of the cage between unlocked or uncovered positions (FIGS. 7A-7E, 8A and 8C) and locked or covered positions (FIGS. 8B and 8D), as shown. In this merely exemplary embodiment, first and second covering members 710a and 710b (labeled in FIG. 7A) are adapted to rotate about locations 712a and 712b (labeled in FIG. 7A) relative to the cage body between the unlocked/uncovered and locked/covered positions relative to the stabilizing member interfaces. FIGS. 8B and 8D illustrate how the covering members, when in their locked/covered positions, minimize or prevent back-out of the stabilizing members in the anterior direction by providing a backstop for the stabilizing members. In this example, the covering members may be referred to as “tabs,” but covering members herein have a variety of configurations and may be adapted to move relative to the cage body in a variety of ways and still help prevent back-out of the stabilizing members.

The systems and methods of use herein include one or more SI joint stabilizing members, which in the embodiments in FIGS. 2, 3A, 3B, 3C and 3D are threaded implants (e.g., screws). The disclosure herein may describe the SI joint stabilizing members as screws in particular embodiments, but it is understood that stabilizing screws herein may be generalized to stabilizing members more generally unless indicated to the contrary. In general, the SI joint stabilizing members (even when they optionally each comprise more than one component coupled in-situ) each have a length from a cage end to an ilium end (measured in the direction “L” shown in FIG. 2, along the stabilizing member axis) that allows the stabilizing members to extend from a fusion cage that is placed between the L5 and S1 vertebrae, through the S1 endplate, through the sacrum, across an SI joint, and into the ilium, to provide stability and/or fusion to the SI joint(s). Additionally, the SI joint stabilizing member cage end (optionally comprising a head, as shown in FIG. 2) is generally configured to interface or engage (optionally stably interface or engage) with the stabilizing member interface of the fusion cage body when fully implanted. The SI joint stabilizing members herein in this context are generally considered to be relatively long such that they can extend from the fusion cage and into the ilium, but generally have a profile that is not large enough to extend into undesired anatomical locations when implanted, such as beyond the lateral cortex of the ilium, the ventral cortex of the sacrum, the S1 foramen, and the sciatic notch.

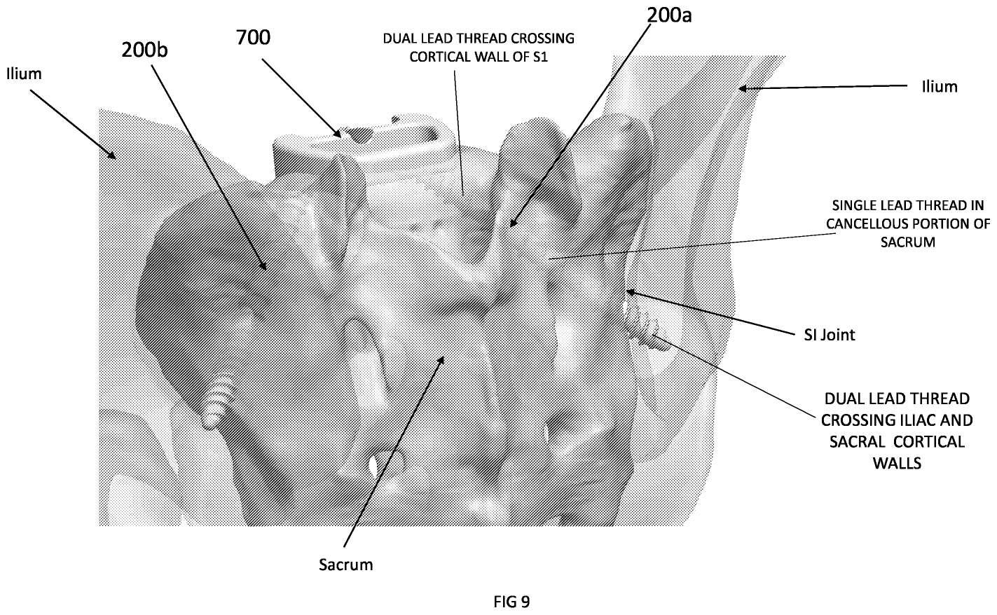

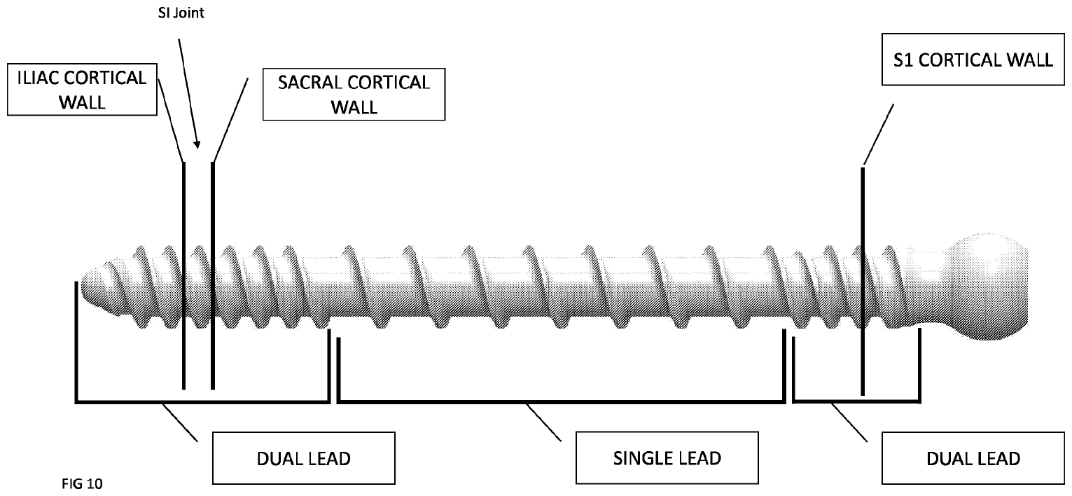

In any of the embodiments herein, a relatively shorter L5 stabilizing member (e.g., threaded cranial screw 300) may have a length from 15 mm to 30 mm, including any subrange or discrete length within that range. In any of the embodiments herein, the relatively shorter L5 stabilizing member may have a diameter from 4.0 mm to 6.5 mm, including any subrange or discrete length within that range. The optional L5 stabilizing member(s) is shorter than the SI joint stabilizing members (e.g., 200a, 200b, FIG. 9, FIG. 10), since it extends into the L5 in a cranial and posterior direction relative to the cage, wherein the SI joint stabilizing members are long enough to extend across the SI joint and into the ilium. The difference is sizes can be seen in, for example, FIG. 2.

In any of the embodiments herein, the relatively longer SI joint stabilizing members (e.g., 200a, 200b, FIG. 9, FIG. 10) may have a length from 60 mm to 100 mm, including any subrange or discrete length within that range. In any of the embodiments herein, the relatively longer caudal stabilizing members may have a diameter from 5.5 mm to 8.0 mm. It is understood that the cross-sectional profile of the stabilizing members herein is not necessarily circular, so the description of exemplary diameters in this context does not limit the configuration of the stabilizing members herein to circular cross sections.

The fusion cages herein also optionally include at least one L5 stabilizing member interface, which can be similar to the SI joint stabilizing member interfaces herein, and all applicable description thereof is incorporated by reference into the L5 stabilizing member interfaces herein. FIG. 2 illustrates an exemplary L5 stabilizing member 300, which in this embodiment is a threaded member (e.g., a screw). The fusion cage, as shown, includes an L5 stabilizing member interface 304 that has a channel configuration that is oriented cranial and posterior relative to the rest of the cage body 102. As shown in FIG. 5, the trajectory of the stabilizing member interface 304 or axis may be such that is facilitates cranial and posterior implantation of the stabilizing member along an axis that is aligned with or substantially aligned with the midline of the fusion cage, as shown in FIG. 3B. As can be seen in FIG. 7E, the channel for the L5 stabilizing member extends from an anterior opening in the cage in a cranial and posterior direction. In the embodiment in FIGS. 7A-8D, an anterior opening to the channel for the L5 stabilizing member is laterally between the covering members 712a and 712b, which is shown in at least FIGS. 7A, 8A, and 8B. This embodiment is also an example of covering members that are configured to cover or block both the L5 stabilizing member and the SI joint stabilizing members, which helps prevent or minimize back-out for both the L5 stabilizing members and the SI joint stabilizing member, which can be seen in the locked/covered position shown in FIG. 8B. Covering members 712a and 712b each include a plurality of protrusions 711 (as shown in FIGS. 7A-8D) that are shaped and spaced such that when the covering members in the unlocked/uncovered positions, the stabilizing members can be advanced and interfaced with the cage, but when the covering members are moved relative to the body, the covering configuration, the protrusions (e.g., elongate and finger-like; L-shaped; boomerang shaped) on each covering member each block one of the stabilizing members, as shown. In embodiments herein, the protrusions or extensions can be considered generally to intersect at an angle less than 180 degrees, as shown.

While stabilizing members may be shown and described herein as threaded, stabilizing members herein may alternatively be non-threaded. For example, any of the stabilizing members herein may be unthreaded and have, for example, circular, oval, or rectilinear cross sectional outer profiles, and which may be implanted with impaction with an axial directed force along an axis. The fusion cages herein may be modified to accommodate the configuration(s) of the stabilizing members.

Each of the stabilizing members herein may have one or more threads at least partially along its length, as shown. FIGS. 9 and 10 illustrate exemplary SI joint stabilizing members (in the form of screws) with exemplary lead configurations, as well as the implanted position of the stabilizing members relative to adjacent tissue (including the SI joint). FIG. 10 illustrates adjacent anatomy conceptually. As shown in the example, the SI joint stabilizing members can each include a dual lead cage region, a single lead central region, and a dual lead ilium region. When implanted, only a distal end of the screw is positioned in the ilium beyond the SI joint, such as no more than 25% of the length of the screw. In this example, as shown in FIGS. 9 and 10, a dual lead section of the screw (including the ilium end) is implanted across the SI joint.

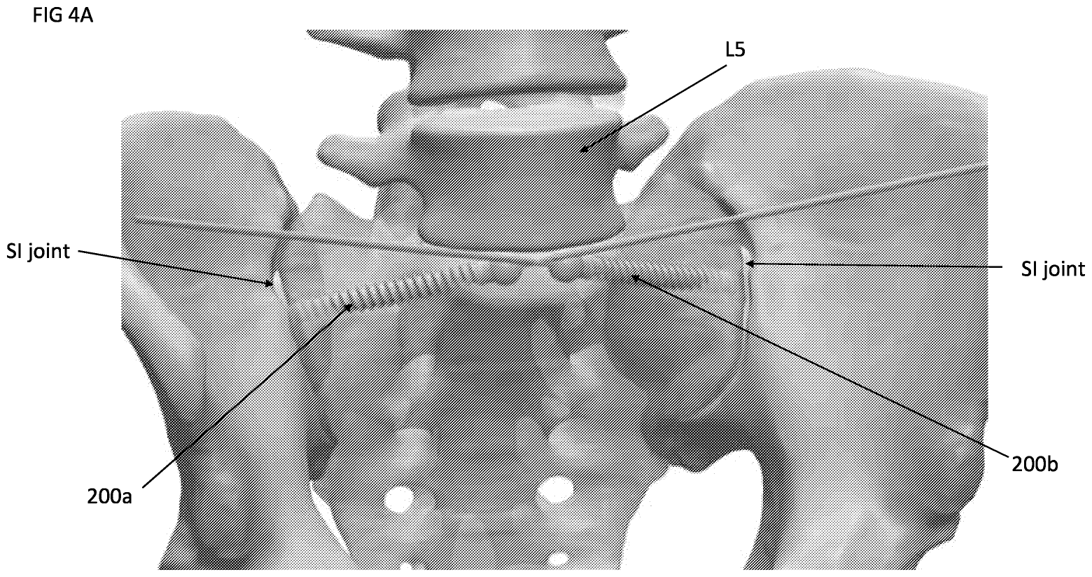

FIGS. 4A, 4B, 4C and 4D illustrate SI joint stabilizing members (FIG. 4D shows exemplary cage 100), as well as optional pins that may be used to help with the trajectory of the screws when implanted, as shown. FIGS. 4A-4D do not illustrate the optional L5 stabilizing member for clarity. The cage is not shown in FIGS. 4A-4C for clarity.

FIGS. 3A, 3B, 3C and 3D (as well as FIG. 5, which includes optional additional treatments/implants) illustrate exemplary fusion systems in exemplary implanted locations. Some anatomical regions are removed from certain figures for clarity. Fusion cage 100 is shown having been implanted between an L5 and S1 vertebrae (between the sacrum and L5 vertebra) from an anterior access, for example. A first stabilizing implant 200a has been implanted such that a cage end region of the first SI joint stabilizing member is engaged with an anterior interface of the fusion cage as shown, and the SI joint stabilizing implant 200a extends across a first SI joint and into a first ilium, as shown. A second SI joint stabilizing implant 200b has been implanted such that a cage region of the second stabilizing implant is engaged with the fusion cage, and extends caudally, posteriorly and laterally across a second SI joint and into a second ilium, as shown. The SI joint stabilizing implants 200a and 200b in FIGS. 3A-3D extend from the fusion cage stabilizing member interfaces, across the S1 endplate, laterally (in different lateral directions) through the sacrum, across the respective SI joint, and into the respective ilium.

When implanting the system, the order of implanting the system components may vary. For example, in some methods, initiating the fusion cage implantation may occur prior in time to (not necessarily immediate before) initiating the implantation of the first and second stabilizing members. In some methods, implanting the first and second stabilizing members may be initiated after the fusion cage is only partially implanted (e.g., ⅔rds implanted) into position and before full fusion cage implantation.

In some methods, initiating implantation of the first and second SI joint stabilizing members may be initiated before initiating implantation of the fusion cage, and the cage can be implanted and positioned to engage the cage end of the stabilizing members.

In any of the methods herein, implanting threaded stabilizing members may comprise rotationally advancing the stabilizing members through the S1 endplate, through the sacrum, across the respective SI joint, and into the respective ilium.

In any of the methods herein, implanting SI joint stabilizing members may comprise advancing the stabilizing members in caudal, lateral and posterior directions towards an SI joint, as shown in the exemplary FIGS. 3A-3D and 5.

FIG. 5 illustrates a view in the caudal direction (top of the fusion cage is shown), and illustrates an optional additional treatment in which an optional additional SI joint stabilizing implant 600 has been implanted from a posterior approach into the sacrum, across the left SI joint, and into the left ilium (a second implant 600′ (not shown) may optionally and similarly be positioned on the right side across the right SI joint). This approach may generally be referred to as an SAI (e.g., S2AI) approach, examples of which are described in PCT publication WO/2020/168269, the disclosure of which is fully incorporated by reference herein for all purposes. Thus, any of the implants described in WO/2020/168269 may be implanted in the manner shown in FIG. 5, and implant 600 may similarly be implanted along any of the trajectories or approaches across an SI joint described in WO/2020/168269. The methods and systems herein may thus be implanted in subjects who have or will have a posterior spinal construct implanted therein, additional exemplary details of which are described in WO/2020/168269. The fusion cage systems herein may thus be part of a more comprehensive overall SI joint stabilization and/or fusion treatment for a subject.

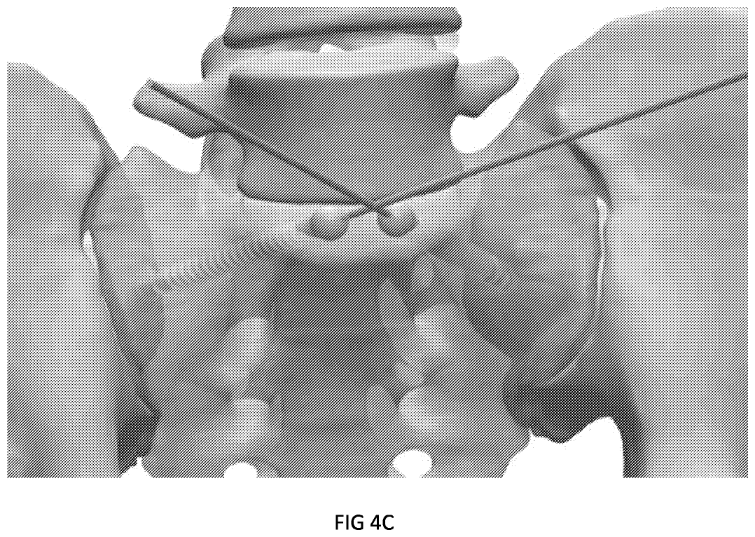

In alternative embodiments, the fusion cages herein and at least cage end regions of the stabilizing members may alternatively be implanted at least partially in the L5 vertebra. FIGS. 6A and 6B illustrate generally an implantation trajectory (optionally over a guide pin) and orientation of the stabilizing members in methods in which the fusion cage is implanted in the L5 vertebra (fusion cage not shown for clarity in FIGS. 6A, 6B and 6C). In methods in which the cage is implanted within the L5 vertebra, a posterior construct (examples of which are described in WO/2020/168269) may be coupled to the fusion cage to help stabilize the fusion cage. While the implants shown in FIGS. 6A, 6B and 6C have rectilinear cross-sectional configurations, and are generally shown and described in US 2018/0228621 (the disclosure of which is incorporated by reference herein in its entirety for all purposes), any of the threaded stabilizing members herein may be implanted in the manner shown in FIGS. 6A, 6B and 6C.

While some examples herein include methods of implanting SI joint stabilizing members from a sacrum-to-ilium approach, in alternative methods of deliver the SI joint stabilizing members may be delivered in an ilium-to-sacrum approach. In these alternative delivery trajectories, the implant can be delivered along the same axis, but is delivered first through the ilium then towards the S1 endplate.

Additionally, while some examples herein include single piece SI joint stabilizing members, the SI joint stabilizing members may be two (or more pieces) that are coupled together in-situ. For example, a first component may be delivered from an ilium-to-sacrum approach, and a second component can be delivered from a sacrum-to-ilium approach, and the two different components can be coupled together during delivery, such as with one component being advanced into a lumen of a second component, and optionally being locked together.

Any description of any of the examples and embodiments herein may be combined with any description of any of the other suitably combinable examples and embodiments herein unless indicated herein to the contrary.

1. A method of fusing vertebrae and stabilizing sacro-iliac joints of a subject, comprising:
implanting at least a portion of a fusion cage between an S1 vertebra and an L5 vertebra;
implanting a first SI joint stabilizing member such that,
a cage end region of the first SI joint stabilizing member interfaces with a first lateral portion of the fusion cage,
the first SI joint stabilizing member extends through an S1 endplate,
an ilium end of the first SI joint stabilizing member is implanted in a first ilium and is posterior, lateral and caudal relative to the fusion cage, and
an intermediate region of the first SI joint stabilizing member that is between the ilium end and the cage end region is implanted across a first SI joint; and
implanting a second SI joint stabilizing member such that,
a cage end region of the second SI joint stabilizing member interfaces with a second lateral portion of the fusion cage,
the second SI joint stabilizing member extends through the S1 endplate,
an ilium end of the second SI joint stabilizing member is implanted in a second ilium and is posterior, lateral and caudal relative to the fusion cage, and
an intermediate region of the second SI joint stabilizing member that is between the ilium end and the cage end region of the second SI joint stabilizing member is implanted across a second SI joint.

2. The method of claim 1, further comprising implanting an L5 stabilizing member such that a cage end region of the L5 stabilizing member interfaces with the fusion cage and an L5 end of the L5 stabilizing member is in the L5 vertebra and cephalad to the fusion cage.

3. The method of claim 2, wherein the cage region of the L5 stabilizing member is laterally between the first and second stabilizing members.

4. The method of claim 1, wherein implanting at least a portion of the fusion cage is initiated prior in time to implanting the first SI joint stabilizing member and implanting the second SI joint stabilizing member.

5. The method of claim 1, wherein implanting the first SI joint stabilizing member comprises implanting the first SI joint stabilizing member in a lateral, caudal and posterior trajectory relative to the fusion cage.

6. The method of claim 5, wherein implanting the second SI joint stabilizing member comprises implanting the second SI joint stabilizing member in a lateral, caudal and posterior trajectory relative to the fusion cage.

7. The method of claim 1, wherein implanting the first and second SI joint stabilizing members comprises rotationally implanting the first and second SI joint stabilizing members.

8. The method of claim 1, wherein implanting the first SI joint stabilizing member comprises advancing the first SI joint stabilizing member from the first ilium and across the first SI joint towards the fusion cage.

9. The method of claim 1, wherein implanting the second SI joint stabilizing member comprises implanting the second SI joint stabilizing member from the second ilium and across the second SI joint towards the fusion cage.

10. The method of claim 1, wherein implanting the first SI joint stabilizing member comprises coupling first and second members of the first SI joint stabilizing member in-situ.

11. The method of claim 10, wherein the first member is delivered through the first sacrum and towards the first ilium, and the second member is delivered from the first ilium and towards the first sacrum.

12. The method of claim 10, wherein implanting the second SI joint stabilizing member comprises coupling first and second members of the second SI joint stabilizing member in-situ.

13. The method of claim 12 wherein the first member is delivered through the second sacrum towards the second ilium, and the second member is delivered from the second ilium and towards the second sacrum.

14. The method of claim 1, further comprising moving a first covering member of the fusion cage from an uncovered position to a covered position in which the first covering member covers at least a portion of the cage end region of the first SI joint stabilizing member.

15. The method of claim 14, further comprising moving a second covering member of the fusion cage from an uncovered position to a covered position in which the second covering member covers at least a portion of the cage end region of the second SI joint stabilizing member.

16. The method of claim 14, wherein moving the first covering member of the fusion cage from an uncovered position to a covered position also covers at least a portion of a cage end region of an L5 stabilizing member.

17. A system for fusing vertebrae and stabilizing sacro-iliac joints of a subject, comprising:
a fusion cage sized and configured to be at least partially implanted between an S1 vertebra and an L5 vertebra;
a first SI joint stabilizing member; and
a second SI joint stabilizing member, wherein
the fusion cage comprising,
a first SI joint stabilizing member interface in an anterior portion and in a first lateral portion of the fusion cage,
the first SI joint stabilizing member interface having at least one surface sized and configured to stably interface with a cage end region of the first SI joint stabilizing member when the first SI joint stabilizing member extends posterior, caudal and lateral relative to the fusion cage such that an ilium end of the first SI joint stabilizing member is implanted in a first ilium of the patient, and
a second SI joint stabilizing member interface in an anterior portion and a second lateral portion of the fusion cage,
the second SI joint stabilizing member interface having at least one surface sized and configured to stably interface with a cage end region of the second SI joint stabilizing member when the second SI joint stabilizing member extends posterior, caudal and lateral relative to the fusion cage such that an ilium end of the second SI joint stabilizing member is implanted in a second ilium of the patient.

18. The system of claim 17, wherein the fusion cage further comprises an L5 stabilizing member interface in the anterior portion of the fusion cage and disposed laterally between the first and SI joint stabilizing member interfaces, the L5 stabilizing member interface sized and configured to stably interface with a cage end region of the L5 stabilizing member when an L5 end of the L5 stabilizing member is in the L5 vertebra and cephalad to the fusion cage.

19. The system of claim 18, wherein the L5 stabilizing member is shorter than the first and second SI joint stabilizing members.

20. The system of claim 19, wherein the L5 stabilizing member has a minor diameter that is less than minor diameters of the first and second SI joint stabilizing members.

21. The system of claim 19, wherein the first and second SI joint stabilizing members each have lengths from 60 mm to 100 mm.

22. The system of claim 17, wherein the first SI joint stabilizing member interface comprises a first channel that is oriented posterior, caudal and lateral relative to a midline of the fusion cage, and wherein the second SI joint stabilizing member interface comprises a second channel that is oriented posterior, caudal and lateral relative to the midline, the midline measured across a width of the fusion cage.

23. The system of claim 22, wherein the first channel is open on an anterior side of the first channel and is closed on a posterior side of the first channel, and wherein the second channel is open on an anterior side of the second channel and is closed on a posterior side of the second channel.

24. The system of claim 17, wherein the first SI joint stabilizing member interface comprises a channel configuration that is oriented laterally away from a midline of the fusion cage, the midline measured across a width of the fusion cage, and wherein the second SI joint stabilizing member interface comprises a second channel configuration that is oriented laterally away from the midline of the fusion cage.

25. The system of claim 17, wherein the first SI joint stabilizing member interface comprises a curved surface that is configured to engage with a curved surface of the cage end region of the first SI joint stabilizing member, and wherein the second SI joint stabilizing member interface comprises a curved surface that is configured to engage with a curved surface of the cage end region of the second SI joint stabilizing member.

26. The system of claim 17, wherein the first and second SI joint stabilizing members each have an intermediate single lead thread region that is axially in between dual lead thread regions.

27. The system of claim 17, wherein the first and second SI joint stabilizing members each have a length from 60 mm to 100 mm.

28. The system of claim 17, wherein the fusion cage further comprises a first covering member moveable from an uncovered position to a covered position in which the first covering member covers at least a portion of the cage end region of the first SI joint stabilizing member.

29. The system of claim 28, wherein the fusion cage further comprises a second covering member moveable from an uncovered position to a covered position in which the second covering member covers at least a portion of the cage end region of the second SI joint stabilizing member.

30. The system of claim 28, wherein in the covered position the first covering member of the fusion cage also covers at least a portion of a cage end region of an L5 stabilizing member.

31. A fusion cage that is adapted for SI joint stabilization, comprising:
a body sized and configured to be at least partially implanted between an S1 vertebra and an L5 vertebra, the body including,
a first SI joint stabilizing member interface in an anterior portion and in a first lateral portion of the fusion cage, and
the first SI joint stabilizing member interface having at least one surface sized and configured to stably interface with a cage end region of a first SI joint stabilizing member when the first SI joint stabilizing member extends posterior, caudal and lateral relative to the body such that an ilium end of the first SI joint stabilizing member is implanted in a first ilium of the patient,
a second SI joint stabilizing member interface in the anterior portion and in a second lateral portion of the fusion cage, and
the second SI joint stabilizing member interface having at least one surface sized and configured to stably interface with a cage end region of a second SI joint stabilizing member when the second SI joint stabilizing member extends posterior, caudal and lateral relative to the body such that an ilium end of the second SI joint stabilizing member is implanted in a second ilium of the patient.

32. The fusion cage of claim 31, further comprising an L5 stabilizing member interface in the anterior portion of the fusion cage and disposed laterally between the first and SI joint stabilizing member interfaces, the L5 stabilizing member interface sized and configured to stably interface with a cage end region of an L5 stabilizing member when an L5 end of the L5 stabilizing member is in an L5 vertebra and cephalad to the body.

33. The fusion cage of claim 32, wherein the L5 stabilizing member interface has a smaller inner dimension than the first and second SI joint stabilizing member interfaces.

34. The fusion cage of claim 32, wherein the L5 stabilizing member interface has an axis that is aligned with a midline of the body, the midline measured across a width of the body.

35. The fusion cage of claim 31, wherein the first and second SI joint stabilizing member interfaces are the only stabilizing member interfaces that have an axis lateral to a midline of the body and that are in the anterior portion of the body.

36. The fusion cage of claim 31, wherein the first and second SI joint stabilizing member interfaces are each oriented such that when SI joint stabilizing members each having a length from 60 mm to 100 mm are interfaced with the stabilizing member interfaces, the SI joint stabilizing members extend further laterally than the body.

37. The fusion cage of claim 31, wherein the fusion cage further comprises a first covering member moveable from an uncovered position to a covered position in which the first covering member covers at least a portion of the first SI joint stabilizing member interface.

38. The fusion cage of claim 37, wherein the fusion cage further comprises a second covering member moveable from an uncovered position to a covered position in which the second covering member covers at least a portion of the second SI joint stabilizing member interface.

39. The fusion cage of claim 37, wherein in the covered position the first covering member of the fusion cage also covers at least a portion of a cage end region of an L5 stabilizing member.
